SEQUENCE LISTING (34 sequences)

SEQ ID NO: 1 - protein, 386 aa, Zea mays L.
MASSSDEQSKPPEPPAAAAVVTAAAPPQTHAEWVASLQAYYAAAGHPYAWPAQHLMAAAAAGAHFGTPVPFPVYHPGAAAAYYAHASMAAGVPYPTCEAVPAVALPTVPEGKGKGKGGGASPEKGSSGAPSGEDASRSDDSGSDESSETRDDDTDHKDSSAPKKRKSGNTSAEGEPSQATVVRYAAVESPYPAKGRSASKLPVSAPGRAALPSATPNLNIGMDIWNASPALAVPAVQGEVSPGLALARRDGVTQLDEREIKRERRKQSNRESARRSRLRKQQECEELARKVADLTTENSALRAELDNLKKACQDMEAENSRLLGGVADAQVPSVTTTLGMSIEPPKLQLQLQQHHDEEGQLHKKSSNNSNGNCAGGSHKPEANTTR

SEQ ID NO: 2 - DNA, 1158 bp, Zea mays L.
atggcgtcgtcctccgacgagcagtccaagccgccggagtcgcccgccgccgccgccgtggtcaccgccgcagcaccgccacagacgcacgccgagtgggtcgcttcgcttcaggcctactacgctgccgcggggcacccctacgcctggccggcgcagcacctcatggcggcggctgcggcgggggcgcacttcggcacgccggtgccgttccccgtctaccacccaggcgccgccgcggcgtactacgcgcacgcgtccatggccgcgggcgtcccttacccgacgtgcgaagctgtccctgcggtggcgctgcccactgtgccggaagggaaagggaagggtaagggcggaggcgcgtcgcctgagaaaggcagctccggggcgccctccggcgaggacgcttctaggagcgatgacagcggcagcgatgagtcatcggagactagagatgatgacactgaccataaggattcatctgcgcccaagaagaggaaatctggtaacacatcggctgaaggtgagccgtctcaagctactgttgtgcgatatgctgcggttgagtcaccatatcccgcaaaaggaaggtctgcctcaaagcttccagtgtctgcacctgggcgtgcagcgcttcctagtgccaccccgaatctaaacattgggatggacatttggaatgcttctcctgccttggctgtgcctgcagtgcagggggaagtgagtcctgggttggcacttgcccgacgtgatggcgttactcaactggacgaacgtgaaataaagagggagaggcgaaaacaatctaacagggagtctgcaaggagatctagattacgcaagcagcaagagtgcgaggagttagcccggaaggtagctgacctaacgaccgagaacagcgctctcagagcagaacttgacaacctcaagaaggcttgtcaagacatggaagcagaaaattcacgtctgttgggtggggtggctgacgcccaggtaccaagtgtcacgaccacactgggaatgagcatcgagccgccgaagttgcagctgcagctgcagcagcatcatgatgaggagggccagctccacaagaaatctagtaataacagcaacgggaactgtgctggaggcagccacaaaccggaggctaacactactagg

SEQ ID NO: 3 - protein, 388 aa, Oryza.sativa L.
MASSSDEQPKPPEPPAAAAVAGTAVATAAAAVPTHAEWAASLQAYYAAAGHPYAWPAQHLMAAAAAGAPYGAPVPFPMYHPGAAAAYYAHASMAAGVPYPTAEAMAAAAAAAAGAVPEGKGKGKGAAASPEKGSSAAPSGDDASRSGDSGSEESSDTRDDDTDHKDSSAPKKRKSGNTSAEGEPSQATLVPYAAVESPYPLKGRSASKLPVSAPGRAALPNATPNLNIGIDLWSTPPALAVPAGQGEASPGLALARRDGVAHLDERELKRERRKQSNRESARRSRLRKQQECEELARKVAELTTENSALRSELDQLKKACEDMEAENTRLMGDKAQYKGPTVTTTLGMSIDSSKTQHHDDEGQLHKNTNNNSNGNYVGGSHKPEANSR

SEQ ID NO: 4 - DNA, 1167 bp, Oryza.sativa L.
atggcgtcctcgtcggacgagcagccgaagccgccggagccgcccgcggcggcggcggtggcggggacggccgtggccaccgccgccgcggcggtgccgacgcacgccgagtgggcggcttcgctgcaggcgtactacgccgccgcggggcacccctacgcgtggcccgcgcagcatctgatggcggcggcggctgcgggggcgccgtacggcgcgccggtgccgttcccgatgtaccacccgggcgccgccgcggcgtactacgcgcacgcgtccatggccgcgggtgttccttacccgacagctgaagccatggcggcggcggcggcggcggcggcgggggcggtgccggaagggaaggggaaggggaagggcgccgccgcgtcgcctgagaagggaagctccgcggcgccctctggggatgatgcatcccggagtggtgacagtggcagcgaggagtcgtctgatactagagatgatgacactgaccacaaggattcgtctgcacctaagaaaaggaaatctggtaatacatcggcagaaggtgagccgtctcaagctacgcttgtgccctatgctgctgtcgagtcaccgtatccgttgaaggggaggtctgcgtcgaagcttccagtttctgcaccagggcgggcggcacttcctaatgccacacctaatttgaacatagggatagatctttggagtactcccccagccttagctgtgcccgcagggcagggggaagcaagtcctgggttggcacttgctcgacgtgatggtgttgctcacctggatgagcgtgaattgaagagggagaggcgcaaacaatctaacagagagtctgccaggagatcaaggttgcgcaagcagcaagagtgtgaggaactagctcggaaggttgctgaactgacaactgagaacagtgcccttcggtcagagcttgatcagcttaagaaggcctgtgaggatatggaagcagagaatacacgactgatgggtgataaggctcaatacaagggaccaactgtgacaaccactctgggtatgagcatcgactcatcgaagacgcaacaccatgacgacgagggccagcttcacaagaacactaataataacagcaacgggaactatgtaggtggcagccacaaaccagaggctaactctaggtga

SEQ ID NO: 5 - DNA, 41 bp, Artificial Sequence
  Primer
gtgttacttctgttgcaacatggcgtcgtcctccgacgagc

SEQ ID NO: 6 - DNA, 49 bp, Artificial Sequence
  Primer
ccatcatggtctttgtagtccctagtagtgttagcctccggtttgtggc

SEQ ID NO: 7 - DNA, 23 bp, Artificial Sequence
  Primer
tcaaatatagcctgcattgttaa

SEQ ID NO: 8 - DNA, 24 bp, Artificial Sequence
  Primer
gtaggatatggggtgtgtttgcca

SEQ ID NO: 9 - DNA, 22 bp, Artificial Sequence
  Primer
agaggacttgaagattgtatgg

SEQ ID NO: 10 - DNA, 19 bp, Artificial Sequence
  Primer
tggcaggcccatcaggtcg

SEQ ID NO: 11 - DNA, 20 bp, Artificial Sequence
  Primer
cagaggataagccgcaccac

SEQ ID NO: 12 - DNA, 19 bp, Artificial Sequence
  Primer
tggcaggcccatcaggtcg

SEQ ID NO: 13 - DNA, 17 bp, Artificial Sequence
  Primer
tgcccstccactccccg

SEQ ID NO: 14 - DNA, 20 bp, Artificial Sequence
  Primer
ggctgaggcaataagaaggg

SEQ ID NO: 15 - DNA, 20 bp, Artificial Sequence
  Primer
ccaaaacccccatcacccaa

SEQ ID NO: 16 - DNA, 21 bp, Artificial Sequence
  Primer
cctatggatggggaggtttgc

SEQ ID NO: 17 - DNA, 20 bp, Artificial Sequence
  Primer
cgagatccacacatccaagg

SEQ ID NO: 18 - DNA, 22 bp, Artificial Sequence
  Primer
gcgctatatccggatggtgggt

SEQ ID NO: 19 - DNA, 21 bp, Artificial Sequence
  Primer
atatcaggaccggaccatacg

SEQ ID NO: 20 - DNA, 18 bp, Artificial Sequence
  Primer
cacaggtgtgaccgccgg

SEQ ID NO: 21 - DNA, 20 bp, Artificial Sequence
  Primer
ccggtgctatcctcttcgag

SEQ ID NO: 22 - DNA, 20 bp, Artificial Sequence
  Primer
cttgacgaacatgccctcct

SEQ ID NO: 23 - DNA, 20 bp, Artificial Sequence
  Primer
agaacacgtgcctcaagacg

SEQ ID NO: 24 - DNA, 20 bp, Artificial Sequence
  Primer
cttgacgaacatgccctcct

SEQ ID NO: 25 - DNA, 17 bp, Artificial Sequence
  Primer
tgcccstccactccccg

SEQ ID NO: 26 - DNA, 20 bp, Artificial Sequence
  Primer
ggctgaggcaataagaaggg

SEQ ID NO: 27 - DNA, 20 bp, Artificial Sequence
  Primer
gacccggagaagaggctgta

SEQ ID NO: 28 - DNA, 20 bp, Artificial Sequence
  Primer
tgtcgaagatggtggtgtgg

SEQ ID NO: 29 - DNA, 20 bp, Artificial Sequence
  Primer
cgagatccacacatccaagg

SEQ ID NO: 30 - DNA, 22 bp, Artificial Sequence
  Primer
gcgctatatccggatggtgggt

SEQ ID NO: 31 - DNA, 21 bp, Artificial Sequence
  Primer
atatcaggaccggaccatacg

SEQ ID NO: 32 - DNA, 18 bp, Artificial Sequence
  Primer
cacaggtgtgaccgccgg

SEQ ID NO: 33 - DNA, 20 bp, Artificial Sequence
  Primer
ggagtactcccccagcctta

SEQ ID NO: 34 - DNA, 20 bp, Artificial Sequence
  Primer
ttgcgcaaccttgatctcct

(19) United States
(12) Patent Application Publication
(10) Pub. No.: US 2023/0054349 A1
(43) Pub. Date: Feb. 23, 2023

(54) Application of EMBP1 Gene or Protein Thereof

(71) Applicant: CAS Center for Excellence in Molecular Plant Sciences, Shanghai (CN)

(72) Inventors: Xinguang Zhu, Shanghai (CN); Perveen Shahnaz, Shanghai (CN); Mingnan Qu, Shanghai (CN); Genyun Chen, Shanghai (CN)

(21) Appl. No.: 17/786,645

(22) PCT Filed: Dec. 14, 2020
(86) PCT No.: PCT/CN2020/136171

§ 371 Date: Jun. 17, 2022

(30) Foreign Application Priority Data
Dec. 20, 2019 (CN) 201911326959.3

(51) Int. Cl. C12N 15/82; C07K 14/415; C12Q 1/6895

(52) U.S. Cl. CPC C12N 15/8261; C07K 14/415; C12Q 1/6895; C12Q 2600/13; C12Q 2600/158

(57) ABSTRACT

Provided is an application of EmBP1 gene or protein thereof, said gene belonging to the bZIP family of zinc finger proteins, the agronomic traits of a plant being significantly improved when the expression of EmBP1 is raised, comprising: regulating the expression of photosynthetic genes, improving photosynthetic efficiency, improving electron transfer efficiency, increasing yield, biomass, plant height, and increasing the number of tillers, etc. The EmBP1 gene can be used as a target to regulate plant agronomic traits, and is applicable to plant breeding.

### RELATED APPLICATIONS

This application is a National Stage Application under 35 U.S.C. 371 of co-pending PCT application PCT/CN2020/013171 designating the United States and filed Dec. 14, 2020; which claims the benefit of CN application number 201911326959.3 and filed Dec. 20, 2019, each of which are hereby incorporated by reference in their entireties.

### TECHNICAL FIELD

The present disclosure relates to the field of plants and agriculture; more particularly, the present disclosure relates to an application of an EMP1 gene or a protein thereof.

### BACKGROUND OF DISCLOSURE

Plants, in particular crops, are important sources of human food and means of production. Almost all human food and many industrial products are directly or indirectly derived from plants. Economic development and deterioration of the ecological environment bring about a reduction in the area of the cultivated land. As the worldwide population grows continuously, how to balance the population growth and the distress of grain shortage has become a worldwide problem, which presents new challenges for both yield and quality of agricultural products. Increasing the yield of plants, especially crops, is a key to the development of human society. Higher plant yield means that more grain, fruit or wood is harvested at the same cultivated land area, providing powerful support for the development of human society. With the expansion of the population and the decrease in the area of the cultivated land, how to grow more grain on a limited cultivated land has always been the research focus of the agricultural workers. At present, conventional breeding cannot meet this requirement, and a variety of means such as molecular biology and molecular marker assisted breeding can be used to help increase crop yield to a maximum extent. Therefore, it is a very important work to study the means of regulating crop plant type and optimizing crop planting.

90% or more dry weight in the crop is directly derived from photosynthesis. Photosynthesis is also known as the most important chemical reaction on the Earth. Therefore, to improve the photosynthetic efficiency and the utilization rate of the light energy of the crops is always the target pursued by the agricultural scientific researcher.

However, the photosynthetic efficiency is a very complex process, including two stages of light reaction and dark reaction. In the prior art, a variety of means have been tried to improve photosynthesis efficiency, wherein the main strategies include reducing the loss of light respiration, increasing the ratio of Rubisco carboxylation and oxidation reaction, transforming C3 plants into C4 plants, and the like. However, the existing strategies in the art are all focused on improving an aspect of the photosynthetic efficiency. The utilization efficiency of the light energy is need to be improved.

At present, screening of the transcription factor that regulates the upstream of a photosynthetic gene can be a new research target. Theoretically, photosynthetic genes can be bound by different transcription factors to affect expression levels. Transcription factors may affect expression of a series of genes by binding to single or different regulatory sequences in the promoter region. However, at present, there are still very few demonstrations for transcription factors to regulate photosynthetic gene expression and affect crop yields. It is urgent to find such regulators that are truly effective.

### SUMMARY OF DISCLOSURE

The purpose of the present disclosure is to provide a novel molecular module affects the stomatal control switch gene, the biological function of which is crucial for improving the economic yield and biomass of drought-resistant rice.

In a first aspect of the present disclosure, there is provided a use of EmBP1 or an up-regulated molecule thereof for: (a) improving agronomic traits of plants, (b) preparing formulations or compositions for improving agronomic traits of plants, or (c) preparing plants with improved agronomic traits; wherein the improved agronomic traits include: (i) increasing photosynthetic efficiency, (ii) regulating the expression of photosynthetic genes, (iii) increasing yield, (iv) increasing biomass, (v) increasing plant height , (vi) increasing the number of tillers; wherein, the EmBP1 includes its homologues.

In a preferable embodiment, the composition includes an agricultural composition.

In another preferable embodiment, the up-regulated molecule includes: an up-regulated molecule that interacts with EmBP1 to increase its expression or activity; or an expression cassette or expression construct (e.g., an expression vector) that overexpresses EmBP1.

In another aspect of the present disclosure, there is provided a method for improving agronomic traits of plants or preparing plants with improved agronomic traits, comprising: increasing the expression or activity of EmBP1 in plants; wherein the improved agronomic traits include: (i) increasing photosynthesis efficiency, (ii) regulating the expression of photosynthetic genes, (iii) increasing yield, (iv) increasing biomass, (v) increasing plant height, (vi) increasing tiller number; wherein, the EmBP1 includes its homologues.

In a preferable embodiment, increasing the expression or activity of EmBP1 includes: regulating with an up-regulated molecule that interacts with EmBP1, thereby increasing the expression or activity of EmBP1; overexpressing EmBP1 in plants.

In another preferable embodiment, the plant includes a plant selected from the following group, or the EmBP1 is from a plant selected from the following group: Gramineae, Brassicaceae, Solanaceae, Leguminosae, Cucurbitaceae, asteraceae, Salicaceae, Moraceae, Myrtaceae, Lycopodiaceae, Selaginellaceae, Ginkgoaceae, Pinaceae, Cycadaceae, Araceae, Ranunculaceae, Platanaceae, Ulmaceae, Juglandaceae, Betulaceae, Actinidiaceae, Malvaceae, Sterculiaceae, Tiliaceae, Tamaricaceae, Rosaceae, Crassulaceae, Caesalpinaceae, Fabaceae, Punicaceae, Nyssaceae, Cornaceae, Alangiaceae, Celastraceae, Aquifoliaceae, Buxaceae, Euphorbiaceae, Pandaceae, Rhamnaceae, Vitaceae, Anacardiaceae, Burseraceae, Campanulaceae, Rhizophoraceae, Santalaceae, Oleaceae, Scrophulariaceae, Pandanaceae, Sparganiaceae, Aponogetonaceae, Potamogetonaceae, Najadaceae, Scheuchzeriaceae, Alismataceae, Butomaceae, Hydrocharitaceae, Triuridaceae, Cyperaceae, Palmae(Arecaceae), Araceae, Lemnaceae, Flagellariaceae, Restionaceae, Centrolepidaceae, Xyridaceae, Eriocaulaceae, Bromeliaceae, Commelinaceae, Pontederiaceae, Philydraceae, Juncaceae, Stemonaceae, Liliaceae, Amaryllidaceae, Taccaceae, Dioscoreaceae, Iridaceae, Musaceae, Zingiberaceae, annaceae, Marantaceae, Burmanniaceae, Chenopodiaceae, or Orchidaceae. Preferably, the homologue of EmBP1 is derived from the plants described in this paragraph.

In another preferable embodiment, Gramineae are selected from (but not limited to): wheat, rice, maize, sorghum, millet, panicum, barley, oat, rye; said Brassicaceae are selected from (but not limited to): rape, Chinese cabbage, arabidopsis; Malvaceae are selected from (but not limited to): cotton, hibiscus rosa-sinensis, hibiscus; Leguminosae are selected from (but not limited to): soybean, Alfalfa; Solanaceae include (but are not limited to): tobacco, tomato, and pepper; Cucurbitaceae include (but are not limited to): pumpkin, watermelon, and cucumber; Rosaceae include (but are not limited to): apple, peach, plum, begonia; Chenopodiaceae are selected from (but not limited to): sugar beet; Asteraceae include (but are not limited to): sunflower, lettuce, asparagus, Artemisia apiacea, Jerusalem artichoke, Stevia rebaudiana; Salicaceae include (but are not limited to): poplar, willow; Myrtaceae include (but are not limited to): eucalyptus, clove, myrtle; Euphorbiaceae include (but are not limited to): rubber tree, cassava, castor; Fabaceae include (but are not limited to): peanut, pea, Astragalus membranaceus. Preferably, the homologue of EmBP1 is derived from the plants described in this paragraph.

In another preferable embodiment, the plant is selected from the following group, or the EmBP1 is from plants comprising the following group: rice (Oryza sativa L.), maize (Zea mays L.), sorghum (Sorghum bicolor L.), millet (Setaria italica L.), panicum (panicum hallii L.), wheat (Triticum aestivum L.), barley (Hordeum vulgare L.), oat (Avera sativa L.), rye (Secale cereale L.), brachypodium stacei, and brachypodium (Brachypodium distachyon).

In another preferable embodiment, the rice is selected from the group consisting of indica rice and japonica rice.

In another preferable embodiment, the EmBP1 is derived from Gramineae or Brassicaceae; for example, from maize, Arabidopsis.

In another preferable embodiment, the plant is Gramineae, and increasing yield or biomass includes: increasing seed weight, increasing seed number, increasing the weight of seeds (including thousand kernel weight), increasing spike number, increasing spikelet number, increasing spike length.

In another preferable embodiment, regulating the expression of photosynthetic genes includes up-regulating the expression of photosynthetic genes.

In another preferable embodiment, the EmBP1 or homologue thereof regulates (including up-regulates) the expression of photosynthetic genes by regulating the promoter of the photosynthetic gene; preferably, EmBP1 or homologue thereof binds to the G-box of the promoter.

In another preferable embodiment, the photosynthetic genes include photosynthetic genes involved in LHC, PSII, PSI, Cyt b6f, ETC, ATPase, CBB cycle and/or Chlorophyll biological pathway; preferably, the photosynthetic genes include PsbR3, RbcS3, FBA1, FBPse, Fd1, PsaN and/or CP29.

In another preferable embodiment, improving photosynthetic efficiency includes: increasing CO₂ absorption rate, increasing electron transfer efficiency, increasing maximum electron transfer rate, increasing Rubisco maximum catalytic efficiency (Vcmax), increasing chlorophyll (including chlorophyll a+b) content, increasing maximum quantum yield (Fv/Fm), increasing the aperture beam size of the reaction center (ABS/RC), and improving the level of the electron transport chain (photosynthetic system I and photosynthetic system II).

In another preferable embodiment, the amino acid sequence of the EmBP1 polypeptide is selected from the group consisting of: (i) a polypeptide having the amino acid sequence shown in SEQ ID NO: 1; (ii) a polypeptide derived from (i) and having one or more (such as 1-50, 1-30, 1-20, 1-10, 1-5, 1-3, 1-2) amino acids deleted, substituted, or inserted in the amino acid sequence of SEQ ID NO: 1, and still having said function of regulating agronomic traits; (iii) a polypeptide with an amino acid sequence having ≥80% (preferably ≥85%, ≥90%, ≥95%, ≥98% or ≥99%) homology to SEQ ID NO:1, and still having said function of regulating agronomic traits; or (iv) an active fragment of the polypeptide of the amino acid sequence shown in SEQ ID NO: 1.

In another preferable embodiment, the nucleotide sequence of the EmBP1 gene is selected from the group consisting of: (a) a polynucleotide encoding the polypeptide shown in SEQ ID NO: 1; (b) a polynucleotide of the sequence shown in SEQ ID NO: 2; (c) a polynucleotide with a nucleotide sequence having ≥80% (preferably ≥85%, ≥90%, ≥95%, ≥98% or ≥99%) homology to SEQ ID NO:2; (d) a polynucleotide formed by truncating or adding 1-60 (preferably 1-30, more preferably 1-10) nucleotides at the 5′ end and/or 3′ end of the polynucleotide shown in SEQ ID NO: 2; (e) a polynucleotide complementary to any of (a)-(d).

In another aspect of the present disclosure, there is provided a plant cell expressing exogenous EmBP1 or its homologue, or an expression cassette comprising exogenous EmBP1 or its homologue; preferably, the expression cassette comprises: promoter, gene encoding EmBP1 or its homologue, terminator; preferably, the expression cassette is contained in a construct or an expression vector.

In another aspect of the present disclosure, there is provided the use of EmBP1 as a molecular marker for identifying agronomic traits of plants; the agronomic traits include: (i) photosynthetic efficiency, (ii) expression of photosynthetic genes, (iii) yield, (iv) biomass, (v) plant height, (vi) tiller number; wherein, the EmBP1 includes its homologues.

In another aspect of the present disclosure, there is provided a method for targeted selection of plants with improved agronomic traits, the method comprising: identifying the expression or activity of EmBP1 in a test plant, if the expression or activity of EmBP1 in the test plant is higher (significantly higher, such as 5% or more, 10% or more, 20% or more, 40% or more, 60% or more, 100% or more or higher) than the average value of the expression or activity of EmBP1 in such plants, then it is an plant with improved agronomic traits; wherein, the improved agronomic traits include: (i) photosynthetic efficiency, (ii) expression of photosynthetic genes, (iii) yield, (iv) biomass, (v) plant height, (vi) tiller number; wherein, the EmBP1 includes its homologues.

Other aspects of the disclosure will be apparent to those skilled in the art based on the disclosure herein.

### BRIEF DESCRIPTION OF DRAWINGS

FIG. 1. Subcellular localization of mEmBP-1a protein;

(a) Schematic diagram of the construction of 35s::mEmBP-1a-EYFP and 35s::NLS-RFP vectors. mEmBP-1a-EYFP fusion protein and NLS-RFP protein (nuclear marker protein) were transiently expressed in rice protoplasts and observed by confocal laser scanning microscopy;

(b) Vector information for transforming the mEmBP-1a gene into rice (japonica Nipponbare); the vector contains the full-length mEmbP-1 a cDNA fused to the FLAG tag and driven by the Ubi-1 promoter and nopaline synthase (nos) terminator (1224 bp);

(c) Relative mRNA expression levels of mEmBP-1a gene in transgenic and wild-type (30 days post-emergence) were analyzed by qRT-PCR. Vertical bars represent the mean ±SE (n=5), and the significance level is expressed by Student's t-test (*P≤0.05; **P≤0.01; ***P≤0.001);

(d) Western data of Nipponbare wild-type rice and EmBP1 transgenic plants;

(e) Picture shows Nipponbare wild-type rice and EmBP1 transgenic rice lines of about 40 DAP in Songjiang Breeding Base, Shanghai;

(e) Picture shows Nipponbare wild-type rice and EmBP1 transgenic rice lines of about 48 DAP in Lingshui Breeding Base, Hainan.

FIG. 2. Leaf photosynthetic physiology measurements of Nipponbare wild-type rice and EmBP1 transgenic rice plants;

(a) Net photosynthetic CO₂ absorption rates (A) at different photosynthetic photon flux densities at a constant [CO₂] of 425 μmolmol⁻¹.

(b) Photosynthetic efficiency at different intercellular CO₂ concentrations under the light intensity of 1800 μmolm⁻²s⁻¹.

(c, d) Photochemical quantum yields of PSII(YII) and PSI(YI) under different PPFD;

(e) Photochemical quenching qL under different PPFD;

(f) redox state of QA(1-qP);

wherein, the values represent mean±SE (n=10).

FIG. 3. Vcmax (Rubisco maximum catalytic efficiency) and maximum electron transfer rate in EmBP1 transgenic lines.

FIGS. 4a-e, Chlorophyll fluorescence parameters in EmBP1 transgenic lines;

FIG. 5. Agronomic traits of Nipponbare wild-type rice and EmBP transgenic rice;

(a) plant height;

(b) tiller number per plant;

(c) Above-ground biomass (shoot biomass) comparison at 50 days post-emergence;

(d) Photographs of field growth 70 days post-emergence in Songjiang Breeding Base, Shanghai (d);

(e) Photographs of field growth 90 days post-emergence in Songjiang Breeding Base, Shanghai;

wherein, vertical bars represent the mean ±SE (n=15), and the significance test is expressed by t-test (*P≤0.05; **P≤0.01; ***P≤0.001);

FIG. 6. Analysis of the phenotype and photosynthetic physiology parameters difference of Arabidopsis thaliana overexpressing maize EmBP1 gene.

(A) The construct of the maize-derived EmBP1 gene expressed by 35s promoter;

(B-C) Compared with wild-type col, both the EmBP1 gene and the encoded protein showed higher expression levels in transgenic lines;

(D) Growth phenotypes of overexpressing-transgenic-lines under climatic chamber conditions.

FIG. 7. Analysis of the phenotype and photosynthetic physiology parameters difference of Arabidopsis thaliana overexpressing maize EmBP1 gene.

FIG. 8. Heat map of expression levels of photosynthesis-related genes in EmBP1 transgenic lines compared to wild-type plants. Data are derived from biological replicates of four different strains. Left penal shows biological pathway names for gene enrichment (GO) analysis.

FIG. 9. Electrophoretic mobility shift assay (EMSA) of the binding of mEmBP-1a to the G-Box motif of target genes in photosynthesis;

(a) Binding assay of mEmBP-1 a and G-Box regulatory element (GCCACGTGGC) of PsbR3 gene;

(b) Binding assay of mEmBP-1 a and G-Box regulatory element (GACACGTGGC) of RbcS3 gene;

(c) Binding assay of mEmBP-1 a and G-Box regulatory element (ATCACGTGTA) of FBA1 gene;

(d) Binding assay of mEmBP-1 a and G-Box regulatory element (GCCACGTGGC) of Fd1 gene;

(e) Binding assay of mEmBP-1 a and G-Box regulatory element (TCCACGTGGC) of PsaN gene;

(f) Binding assay of mEmBP-1a and G-Box regulatory element (TCCACGTGTC) of CP29 gene;

(g) Relative expression levels of each photosynthetic regulatory gene in Nipponbare wild-type rice and EmBP1 transgenic lines under low light (200PPFD);

(h) Relative expression levels of each photosynthetic regulatory gene in Nipponbare wild-type rice and EmBP1 transgenic lines under weak light (500PPFD).

FIG. 10. The phenotypes of the 35S::EmBP1 overexpression line at the mature stage under normal conditions.

A. Imaging of wild-type and three 35S::EmBP1-GFP overexpression lines under normal conditions;

B. Grain weight per plant under normal conditions;

C. Gene expression of OsEmBP1 in overexpression lines.

D. A1200 of the overexpressing line (photosynthesis rate at 1200 light intensity).

Data were obtained from 20 biological replicates for grain weight measurements, and 4 biological replicates for gene expression experiments.

FIG. 11. Gene conservation studies.

A. Using the neighbor-joining method (Saitou and Nei, 1987), a phylogenetic tree of bZIP proteins EmBP-1 of different model plant was constructed with MEGA5; the numbers on the phylogenetic diagram show the bootstrap values of each node; nodes with bootstrap trust degree less than 40% have been collapsed;

B. Homology comparison of amino acids between rice- and maize-derived EmBP1, the same amino acid residues are shown as “*”; the deletion/insertion of amino acids is shown as “-”; “.” or “:” indicates an altered amino acid.

### DETAILED DESCRIPTION

Through extensive research and screening work, the inventors have revealed for the first time an Em Binding Protein, which belongs to zinc finger protein bZIP family, and is encoded by EmBP1 gene. When the expression of EmBP1 gene is increased, the agronomic traits of plants can be significantly improved, including: (i) increasing yield, (ii) increasing biomass, (iii) increasing plant height, (iv) increasing tiller number, (v) regulating expression of photosynthesis genes, (vi) increasing photosynthetic efficiency, (vii) increasing electron transfer efficiency, etc. Therefore, EmBP1 gene can be used as a target for regulating plant agronomic traits and used in plant breeding.

### Genes, Polypeptides, Constructs and Plants

Through the previously constructed photosynthetic gene co-expression regulatory network, the inventors found that EmBP1 can interact with 43 photosynthetic genes. The analysis showed that the number of photosynthetic genes interacting with EmBP1 reached a very significant level (P<0.001), indicating that EmBP1 is very likely to be a key transcription factor regulating photosynthetic efficiency. Transcriptome analysis shows that the photosynthetic efficiency biological pathway, including 65 photosynthetic genes, was significantly enriched in plants overexpressing EmBP1. Among them, the promoter regions of 20 genes have G-BOX regulatory sequences. qPCR results showed that 6 genes were significantly different in overexpressing and wild-type plant material. Further, the inventors confirmed the binding relationship between EmBP1 and some of the photosynthetic genes by Electron mobility shift assay (EMSA). The inventors found that the plant material overexpressing EmBP1 has higher photosynthetic efficiency and electron transfer efficiency, and can also significantly improve plant height, tiller number, grain number and biomass. More importantly, the yield per plant can be increased by 20-30%, indicating the significant value of this gene in high-light-efficiency plant breeding.

As used herein, the terms “EmBP1 of the disclosure”, “EmBP-1a” are used interchangeably. The EmBP1 protein can have the protein (polypeptide) of the amino acid sequence shown in SEQ ID NO: 1, and the gene encoding it can have the nucleotide sequence shown in SEQ ID NO: 2, and the EmBP1 protein also includes its homologues.

As used herein, the terms “mEmBP1 gene of the present disclosure”, “mEmBP-1a gene” are used interchangeably, and both refer to the mEmBP1 gene derived from the crop maize or a variant thereof.

The present disclosure also includes fragments, derivatives and analogs of EmBP1. As used herein, the terms “fragments”, “derivatives” and “analogues” refer to polypeptide that basically maintain the same biological function or activity of EmBP1 of the disclosure. The fragments, derivatives or analogs of the polypeptide in the disclosure may be (i) a polypeptide with one or more (e.g. 1-50, 1-40, 1-30, 1-20, 1-10, 1-5, 1-3, 1-2) conservative or non-conservative amino acid substitution (preferably conservative), where the substituted amino acid residues may or may not be one encoded by the genetic code, (ii) a polypeptide having substituent(s) in one or more (e.g. 1-50, 1-40, 1-30, 1-20, 1-10, 1-5, 1-3, 1 -2) amino acid residues, or (iii) a polypeptide formed by having said polypeptide fused with additional amino acid sequence (such as leader sequence or secretory sequence, or sequence used for purification of the polypeptide or proprotein sequence, or fusion protein). In accordance with the teachings provided herein, these fragments, derivatives and analogs are well known to a person skilled in the art.

Any biologically active fragment of EmBP1 can be used in the present disclosure. Herein, a biologically active fragment of EmBP1 refers to a polypeptide that still retains all or part of the functions of the full-length EmBP1. Typically, the biologically active fragment retains at least 50% of the activity of full-length EmBP1. Under more preferred conditions, the active fragment is capable of retaining 60%, 70%, 80%, 90%, 95%, 99%, or 100% of the activity of full-length EmBP1.

In the present disclosure, EmBP1 also includes a variant form of the sequence of SEQ ID NO: 1 that has the same function as EmBP1. These variations include but are not limited to: deletion, insertion and/or substitution of several (usually 1-50, preferably 1-30, more preferably 1-20, most preferably 1-10, still more preferably 1-8, 1-5) amino acids, and addition or deletion of one or several (usually 1-50, preferably 1-30, more preferably 1-20, most preferably 1-10, still more preferably 1-8, 1-5) amino acids at the C-terminal and/or N-terminal (especially N-terminal). For example, substitution with amino acids of comparable or similar properties usually does not change protein function in the art. As another example, addition of deletion of one or more amino acids to the C-terminal and/or N-terminal (especially N-terminal) usually does not change the function of a protein either.

Any protein having high homology with the EmBP1 (for example, having 60% or higher, 70% or higher, 80% or higher; preferably, 85% or higher; more preferably, 90% or higher homology, such as 95%, 98% or 99% homology with the sequence shown in SEQ ID NO: 1), and having the same function as EmBP1 are also included in the present disclosure. The term “homology” refers to the level of similarity (i.e. sequence similarity or identity) between two or more nucleic acids or polypeptides according to the percentage of identical positions. Herein, variants of the genes can be obtained by inserting or deleting regulatory regions, performing random or site-directed mutagenesis, and the like.

It should be understood that although the EmBP1 of the present disclosure is preferably obtained from maize, other polypeptides or genes obtained from other plants (especially plants belonging to the same family or genus as maize) that highly homologous (such as having more than 60%, such as 70%, 75%, 80%, 85%, 90%, 95%, 98%, or even 99% sequence identity) to the EmBP1 of maize are also within the scope of the present disclosure, as long as those skilled in the art can easily separate and obtain the polypeptides or genes from other plants according to the information provided by the present application after reading the present application. These polypeptides or genes are also referred to as “homologs” of EmBP1. Methods and tools for aligning sequence identity are also well known in the art, such as BLAST.

The present disclosure also relates to polynucleotide sequences encoding the EmBP1 or conservative variant polypeptides of the present disclosure. The polynucleotide can be in the form of DNA or RNA. Forms of DNA include cDNA, genomic DNA or artificially synthesized DNA. DNA can be single-stranded or double-stranded. The DNA may be coding strand or non-coding strand. The sequence encoding the mature polypeptide may be identical to the coding region sequence as shown in SEQ ID NO: 2 or a degenerate variant thereof. “Degenerate variant” used in the disclosure refers to a nucleic acid sequence that encodes the protein of SEQ ID NO: 1, but is different from the coding region sequence shown in SEQ ID NO: 2. Due to the codon degeneracy, even if a polynucleotide sequence having low homology to SEQ ID NO: 2 can basically encoded the amino acid sequence shown in SEQ ID NO: 1.

The polynucleotide encoding the mature polypeptide of SEQ ID NO: 1 includes: the coding sequence only encoding the mature polypeptide; the coding sequence encoding the mature polypeptide and a various additional coding sequence; the coding sequence encoding the mature polypeptide (and an optional additional coding sequence) and a noncoding sequence. The term “polynucleotide encoding a/the polypeptide” can include a polynucleotide encoding the polypeptide, or a polynucleotide that further includes additional coding and/or non-coding sequences.

The disclosure also relates to a vector containing the polypeptide and a host cell generated by genetic engineering with the vector or EmBP1 coding sequence.

The transformation of host cells with recombinant DNA can be carried out by conventional techniques well known to those skilled in the art. Agrobacterium transformation or biolistic transformation and other methods can be used to transform plants, such as spraying method, leaf disk method, immature embryo transformation method and the like.

As used herein, the “plant” is a plant containing a photosynthetic reaction system (including photosynthetic genes involved in photosynthesis), and containing EmBP1 or a homologue thereof. Preferably, the “plants” include (but are not limited to) a plant of: Gramineae, Brassicaceae, Solanaceae, Leguminosae, Cucurbitaceae, asteraceae, Salicaceae, Moraceae, Myrtaceae, Lycopodiaceae, Selaginellaceae, Ginkgoaceae, Pinaceae, Cycadaceae, Araceae, Ranunculaceae, Platanaceae, Ulmaceae, Juglandaceae, Betulaceae, Actinidiaceae, Malvaceae, Sterculiaceae, Tiliaceae, Tamaricaceae, Rosaceae, Crassulaceae, Caesalpinaceae, Fabaceae, Punicaceae, Nyssaceae, Cornaceae, Alangiaceae, Celastraceae, Aquifoliaceae, Buxaceae, Euphorbiaceae, Pandaceae, Rhamnaceae, Vitaceae, Anacardiaceae, Burseraceae, Campanulaceae, Rhizophoraceae, Santalaceae, Oleaceae, Scrophulariaceae, Pandanaceae, Sparganiaceae, Aponogetonaceae, Potamogetonaceae, Najadaceae, Scheuchzeriaceae, Alismataceae, Butomaceae, Hydrocharitaceae, Triuridaceae, Cyperaceae, Palmae(Arecaceae), Araceae, Lemnaceae, Flagellariaceae, Restionaceae, Centrolepidaceae, Xyridaceae, Eriocaulaceae, Bromeliaceae, Commelinaceae, Pontederiaceae, Philydraceae, Juncaceae, Stemonaceae, Liliaceae, Amaryllidaceae, Taccaceae, Dioscoreaceae, Iridaceae, Musaceae, Zingiberaceae, annaceae, Marantaceae, Burmanniaceae, Chenopodiaceae, or Orchidaceae. More preferably, the plant can be Gramineae, such as Oryza L. (such as rice (Oryza sativa L.)), Triticum L. (such as wheat (Triticum aestivum L.)), Zea L. (such as maize (Zea mays L.)) and the like. The EmBP1 or its homologues in the present disclosure can also be derived from plants including the above described plants.

### Methods and Use of Improving Plants

The present disclosure also provides a method for improving a plant, the method comprising increasing the expression of EmBP1 in the plant. Improving a plant includes: (i) increasing photosynthetic efficiency, (ii) regulating the expression of photosynthetic genes, (iii) increasing yield, (iv) increasing biomass, (v) increasing plant height , (vi) increasing the number of tillers. After knowing the function of the EmBP1, various methods well known to those skilled in the art can be used to increase the expression of EmBP1. For example, an expression unit (such as an expression vector or virus, etc.) carrying EmBP1 gene can be delivered to the target through a route known to those in the art, and the active EmBP1 can be expressed.

Preferably, a method for preparing a transgenic plant is provided, comprising: (1) transferring an exogenous EmBP1-encoding polynucleotide into a plant tissue, organ or tissue, and obtaining a plant tissue, organ or seed transformed with the EmBP1-encoding polynucleotide; and (2) regenerating the plant tissue, organ or seed obtained in step (1) transformed with the exogenous EmBP1-encoding polynucleotide into a plant.

Other methods of increasing the expression of the EmBP1 gene or its homologous genes are known in the art. For example, the expression of the EmBP1 gene or its homologous gene can be enhanced by driving with a strong promoter. Alternatively, the expression of the EmBP1 gene can be enhanced by an enhancer (eg, the first intron of waxy gene of rice, the first intron of Actin gene, etc.). Strong promoters suitable for the method of the present disclosure include, but are not limited to: Ubi promoter of rice or maize, 35s promoter, and the like.

The methods can be carried out using any suitable conventional means, including reagents, temperature, pressure conditions, and the like.

Those skilled in the art know that the photosynthesis mechanism of various plants (especially higher plants) is similar, that is: under the irradiation of visible light, light energy is converted into unstable chemical energy by the photosynthetic pigments (mainly chlorophyll a and chlorophyll b) through photoreaction; and then through dark-reaction, the plant converts carbon dioxide and water into stable organic matter, and releases oxygen. Key players in this process include some photosynthetic genes involved in LHC, PSII, PSI, Cyt b6f, ETC, ATPase, CBB cycle and/or Chlorophyll biological pathways, which are conserved in a wide variety of plants. The EmBP1 protein or its encoding gene of the present disclosure can regulate the expression of multiple photosynthetic genes and promote their expression. In the further research of the present inventors, it has been found that the EmBP1 or its homologues can regulate (including up-regulate) the expression of photosynthetic genes by regulating the promoters of the photosynthetic genes; preferably, EmBP1 binds to the G-box of the photosynthetic gene promoters. Given that the G-box is conserved in the promoters of photosynthetic genes, it can be expected that the EmBP1 of the present disclosure or its homologues can play a regulatory role in a variety of plants. Therefore, it should be understood that the technical solutions of the present disclosure can be applied to a variety of plants and are not limited to rice or Arabidopsis specifically listed in the examples.

In addition, the present disclosure also relates to the use of EmBP1 or its encoding gene as a tracking marker for the progeny of genetically transformed plants. The present disclosure also relates to the use of EmBP1 or its encoding gene as a molecular marker to identify the agronomic traits of plants by detecting the expression of EmBP1 in plants. When evaluating the plant to be tested, the expression or mRNA level of EmBP1 can be determined to assess whether the expression or mRNA level in the plant to be tested is higher than the average of such plants. If it is significantly higher, the plant has improved agronomic traits.

Based on the molecular mechanism of the present disclosure and the genes or proteins involved in the molecular mechanism, substances that can be useful in improving agronomic traits of plants can be screened based on the new findings.

The methods for screening substances acting on a protein or a specific region thereof as a target are well known to those skilled in the art, and these methods can be applied to the present disclosure. The candidate substances can be selected from: peptides, polymeric peptides, peptidomimetics, non-peptide compounds, carbohydrates, lipids, antibodies or antibody fragments, ligands, small organic molecules, small inorganic molecules, nucleic acid sequences, and the like. Depending on the type of substances to be screened, it is routine to those skilled in the art how to select a suitable screening method.

The detection of protein-protein interactions and the strength of the interactions can be performed using a variety of techniques well-known to those skilled in the art, such as GST sedimentation technology (GST-Pull Down), bimolecular fluorescence complementation experiments, yeast two-hybrid system or co-immunoprecipitation, etc.

### Main Advantages of the Disclosure

(1) The present disclosure screened for the first time a maize-derived zinc finger protein bZIP family (mEmBP1) gene, which is a transcription factor that can affects the G-BOX regulatory sequence of the promoter regions of multiple photosynthetic genes (such as 6 genes in Example), thereby changing the expression level of genes, affecting the photosynthetic efficiency, the quantum efficiency of the photosynthetic system and the maximum electron transfer efficiency. The technical solution of the present disclosure is superior to the previous improved system by overexpressing a single photosynthetic gene, such as FBPase, SBPase and Rubisco small subunit.

(2) The present disclosure finds for the first time that increasing the expression of EmBP1 gene or its protein can significantly improve the agronomic traits of plants, such as increasing biomass, increasing tiller number, increasing yield per plant, and increasing plant height, etc. The examples of the present disclosure demonstrate that yield per plant can be increased by 10-20%.

(3) By genetic engineering using EmBP1, The present disclosure can influence photosynthetic efficiency genes on the whole, promote plants to adapt to different light environments, improve plant photosynthetic efficiency, increase yield or biomass and the like.

The disclosure is further illustrated by the specific examples described below. It should be understood that these examples are merely illustrative, and do not limit the scope of the present disclosure. The experimental methods without specifying the specific conditions in the following examples generally used the conventional conditions, such as those described in J. Sambrook, Molecular Cloning: A Laboratory Manual (3rd ed. Science Press) or followed the manufacturer's recommendation.

### Materials and Methods

1. Vector Construction and Transgenic Plant Generation

First, mEmBP-1a gene (GRMZM2G095078, full length of 1158 bp) was amplified based on maize B73 by primers (forward: GTGTTACTTCTGTTGCAACATGGCGTCGTCCTCCGACG AGC (SEQ ID NO:5); reverse: CCATCATGGTCTTTGTAGTCCCTAGTAGTGTTAGCC TCCGGTTTGTGGC (SEQ ID NO:6)). The amplified product was ligated to the improved pCAMBIA1302 vector (pCAMBIA1302 vector inserted with Flag and ubi1). The vector contained the EmBP1 gene at 4323-5480 bp with the promoter of ubiquitin (ubi1), and the downstream 5481-5546 bp contained a Flag tag. The EmBP-1a gene was ligated into the pCAMBIA1302 vector, which was transformed into DH5α E.coli, and then the rice Nipponbare was transformed with the vector through Agrobacterium strain LBA4404 and regenerated. Progeny was screened based on the hygromycin resistance to obtain positive seedlings. Finally, the gene expression levels of the three transgenic T₃ progeny lines were identified by qPCR, and the expression level of EmBP1 protein was verified by immunohybridization with Flag antibody.

2. Transgenic Line Growth Conditions

EmBP1-overexpressing rice lines were grown in artificial climate chambers, Shanghai Songjiang Breeding Base and Hainan Lingshui Breeding Base, and their field performance was evaluated.

Climate chamber conditions: rice lines were grown in pot with natural light and watered twice a week. Photosynthetic assays were performed 60 days after sowing. The lines were grown at the room temperature of 27° C., the light intensity of about 600PPFD, the relative humidity of 62-75%, and the photoperiod of 16 hours. Each line has 4 biological replicates.

Shanghai Songjiang Breeding Base and Hainan Lingshui Breeding Base: Longitude and latitude are (121°8′1″ E, 30°56′44″ N) and (110.0375°E, 18.5060°N), respectively. The lines were planted in May 2017 and December 2017. The average temperature during the growing season is about 25° C. and 31° C., respectively.

Shanghai Songjiang Breeding Base and Hainan Lingshui Breeding Base: Longitude and latitude are (121°8′1″ E, 30°56′44″ N) and (110.0375°E, 18.5060°N), respectively. The lines were planted in May 2017 and December 2017. The average temperature during the growing season is about 25° C. and 31° C., respectively.

Six T3 transgenic lines (49 strains per line) were identified by PCR with hygromycin at seedling stage. Three lines (100% positive) were selected, and 10 strains were randomly selected. The protein and gene expression levels were verified by Anti-flag antibody and qPCR respectively.

3. Determination of Photosynthetic Physiological and Biochemical Indicators

Photosynthetic efficiency was measured by a portable photosynthesis instrument (LICOR-6400XT). The temperature of the leaf chamber is 25° C., the light intensity is firstly 1500PPFD, and the CO₂ is 400 ppm. The photosynthetic CO₂ and photosynthetic light intensity reaction curves are made with reference to Chang et al. 2017, and the chlorophyll fluorescence induction curve is completed by M-PEA. Before the measurement, the leaves were dark-adapted for 60 minutes, and the specific procedure was referred to Hamdani et al. 2015. For photosystem I and photosystem II related parameters, including photochemical quenching (qL), photochemical quantum yield (YII), and Qa redox state (1-qP), the determination method was referred to Schreiber and Klughammer 2008. Protein immunohybridization method was referred to He and Mi 2016.

4. Transcriptome Data Determination

The leaves were selected from rice samples at the booting stage from 9:30 am to 11:00 am. After the leaves were quickly frozen with liquid nitrogen, they were stored in a −80° refrigerator for later use. Then, mRNA was extracted by the kit (according to the instructions of PureLink RNA Mini Kit, Life Technologies Corporation), and the degree of mRNA integration was detected. Samples were sequenced by Agilent 2100 Bioanalyzer. Transcriptome data was analyzed by STAR57 software and assembled based on the rice standard genome IRGSP-1.0 version. Annotated genes were characterized by RPKM values, and differentially expressed genes (DEGs) were analyzed by STAR. The up- and down-regulated DEGs were defined using log2 as the standard, respectively.

5. Electrophoretic Mobility Shift Assay (EMSA)

In order to verify the ability of the EmBP1 gene to interact with key photosynthetic genes, the electrophoretic mobility shift assay was performed. Specific methods were referred to Zhai et al. (2019). For key photosynthetic genes, photosynthetic genes whose promoters contain G-BOX were screened, including: Os11g0171300 (FBA1), Os12g0291100 (Rbcs3), Os08g0104600 (Fd1), Os07g0558400 (Lhcb4/CP29), Os12g0189400 (PsaN) and Os08g0200300 (PsbR3). PCR forward primer (with cy5 fluorescent probe sequence): Cy5-TCAAATATAGCCTGCATTGTTAA (SEQ ID NO:7); reverse primer: GTAGGATATGGGGTGTGTTTGCCA (SEQ ID NO:8). The binding solution includes 1 nM Cy5-labeled DNA samples, EmBP1 protein at different concentrations. The nickel-column protein purification was referred to He and Mi 2016, incubated at 4° C. for 1 hour. The reaction system includes 10 mM Tris-HCl (pH 8.0), 0.1 mg/ml BSA, 50 μM ZnCl₂, 100 mM KCl, 10% glycerol, 0.1% NP-40 and 2 mM β-mercaptoethanol. The gel mobility shift assay was performed in 4% non-denaturing gel at 200V for 15 minutes at 4° C. with the solution of 1×Tris—glycine solution (pH 8.3). Images were analyzed by Starion FLA-9000 (FujiFlim, Japan).

TABLE 2
Probe sequences used in EMSA

| | Sequence | SEQ ID NO: |
|---|---|---|
| Os11g0171300-F | AGAGGACTTGAAGATTGTATGG | 9 |
| Os11g0171300-R | TGGCAGGCCCATCAGGTCG | 10 |
| Osl2g0291100-F | CAGAGGATAAGCCGCACCAC | 11 |
| Osl2g0291100-R | TGGCAGGCCCATCAGGTCG | 12 |
| Os08g0104600-F | TGCCCSTCCACTCCCCG | 13 |
| Os08g0104600-R | GGCTGAGGCAATAAGAAGGG | 14 |
| Gs07g0558400-F | CCAAAACCCCCATCACCCAA | 15 |
| Os07g0558400-R | CCTATGGATGGGGAGGTTTGC | 16 |
| Os12g0189400-F | CGAGATCCACACATCCAAGG | 17 |
| Osl2g0189400-R | GCGCTATATCCGGATGGTGGGT | 18 |
| Os08g0200300-F | ATATCAGGACCGGACCATACG | 19 |
| Gs08g0200300-R | CACAGGTGTGACCGCCGG | 20 |

6. Detection of Relative Expression Levels of EmBP1-Targeted Photosynthetic Genes

Rice leaves 5 weeks after emergence were selected, and the samples were stored in liquid nitrogen. RNA extraction was performed with TRIzol Plus RNA purification kit (Invitrogen Life Technology Co., Ltd.), and the operation was performed according to the standard procedure in the instruction. Reverse Transcription to cDNA was performed using SuperScript VILO cDNA Reverse Transcription Kit (Invitrogen Biotech Co., Ltd.). 2 ug of total RNA was used for reverse transcription to cDNA. Quantitative PCR was performed using SYBR Green PCR reaction system (Applied Biosystems, USA) and ABI quantitative PCR instrument (StepOnePlus). The amplification reaction program was: 95° C. for 10 s, 55° C. for 20 s, and 72° C. for 20s. The housekeeping gene is actin. Three biological replicates and three technical replicates were performed. The newly developed primer sequences are as follows (Table 3).

TABLE 3
Primer sequence list of qPCR

| Photo- | | | |
|---|---|---|---|
| synthetic | | | Product |
| Gene ID | | Sequence (SEQ ID NO:) | size |
| Os11g0171300 | F | CCGGTGCTATCCTCTTCGAG (21) | 160 |
| FBA1 | R | CTTGACGAACATGCCCTCCT (22) | |
| Os12g0291100 | F | AGAACACGTGCCTCAAGACG (23) | 139 |
| Rbcs3 | R | CTTGACGAACATGCCCTCCT (24) | |
| Os08g0104600 | F | TGCCCSTCCACTCCCCG (25) | 116 |
| Fd1 | R | GGCTGAGGCAATAAGAAGGG (26) | |
| Os07g0558400 | F | GACCCGGAGAAGAGGCTGTA (27) | 135 |
| Lhcb4/CP29 | R | TGTCGAAGATGGTGGTGTGG (28) | |
| Os12g0189400 | F | CGAGATCCACACATCCAAGG (29) | 100 |
| PsaN | R | GCGCTATATCCGGATGGTGGGT | |
| | | (30) | |
| Os08g0200300 | F | ATATCAGGACCGGACCATACG (31) | 184 |
| PsbR3 | R | CACAGGTGTGACCGCCGG (32) | |

### EXAMPLE 1. THE ACQUISITION OF GENE AND ITS INFORMATION

After large-scale research and screening, the inventors screened for the first time a zinc finger protein derived from maize, which is a gene of the bZIP family and a transcription factor called EmBP1 (mEmBP1) gene.

mEmBP1 protein sequence is as follows
(SEQ ID NO:1)
MASSSDEQSKPPEPPAAAAVVTAAAPPQTHAEWVASLQAYYAAAGHPYA
WPAQHLMAAAAAGAHFGTPVPFPVYHPGAAAAYYAHASMAAGVPYPTCE
AVPAVALPTVPEGKGKGKGGGASPEKGSSGAPSGEDASRSDDSGSDESS
ETRDDDTDHKDSSAPKKRKSGNTSAEGEPSQATVVRYAAVESPYPAKGR
SASKLPVSAPGRAALPSATPNLNIGMDIWNASPALAVPAVQGEVSPGLA
LARRDGVTQLDEREIKRERRKQSNRESARRSRLRKQQECEELARKVADL
TTENSALRAELDNLKKACQDMEAENSRLLGGVADAQVPSVTTTLGMSIE
PPKLQLQLQQHHDEEGQL

The sequence of the coding region of the maize EmBP1 gene is as follows (SEQ ID NO: 2):

>Chr7: 19265565..19270520
ATGGCGTCGTCCTCCGACGAGCAGTCCAAGCCGCCGGAGTCGCCCGCCG
CCGCCGCCGTGGTCACCGCCGCAGCACCGCCACAGACGCACGCCGAGTG
GGTCGCTTCGCTTCAGGCCTACTACGCTGCCGCGGGGCACCCCTACGCC
TGGCCGGCGCAGCACCTCATGGCGGCGGCTGCGGCGGGGGCGCACTTCG
GCACGCCGGTGCCGTTCCCCGTCTACCACCCAGGCGCCGCCGCGGCGTA
CTACGCGCACGCGTCCATGGCCGCGGGCGTCCCTTACCCGACGTGCGAA
GCTGTCCCTGCGGTGGCGCTGCCCACTGTGCCGGAAGGGAAAGGGAAGG
GTAAGGGCGGAGGCGCGTCGCCTGAGAAAGGCAGCTCCGGGGCGCCCTC
CGGCGAGGACGCTTCTAGGAGCGATGACAGCGGCAGCGATGAGTCATCG
GAGACTAGAGATGATGACACTGACCATAAGGATTCATCTGCGCCCAAGA
AGAGGAAATCTGGTAACACATCGGCTGAAGGTGAGCCGTCTCAAGCTAC
TGTTGTGCGATATGCTGCGGTTGAGTCACCATATCCCGCAAAAGGAAGG
TCTGCCTCAAAGCTTCCAGTGTCTGCACCTGGGCGTGCAGCGCTTCCTA
GTGCCACCCCGAATCTAAACATTGGGATGGACATTTGGAATGCTTCTCC
TGCCTTGGCTGTGCCTGCAGTGCAGGGGGAAGTGAGTCCTGGGTTGGCA
CTTGCCCGACGTGATGGCGTTACTCAACTGGACGAACGTGAAATAAAGA
GGGAGAGGCGAAAACAATCTAACAGGGAGTCTGCAAGGAGATCTAGATT
ACGCAAGCAGCAAGAGTGCGAGGAGTTAGCCCGGAAGGTAGCTGACCTA
ACGACCGAGAACAGCGCTCTCAGAGCAGAACTTGACAACCTCAAGAAGG
CTTGTCAAGACATGGAAGCAGAAAATTCACGTCTGTTGGGTGGGGTGGC
TGACGCCCAGGTACCAAGTGTCACGACCACACTGGGAATGAGCATCGAG
CCGCCGAAGTTGCAGCTGCAGCTGCAGCAGCATCATGATGAGGAGGGCC
AGCTCCACAAGAAATCTAGTAATAACAGCAACGGGAACTGTGCTGGAGG
CAGCCACAAACCGGAGGCTAACACTACTAGG

### EXAMPLE 2. ANALYSIS OF PHOTOSYNTHETIC GENE REGULATORY FACTORS BY CO-EXPRESSION REGULATORY NETWORK SCREENING

According to previous studies, the metabolic pathways related to photosynthetic efficiency in Arabidopsis model species were collected from the KEGG database. These genes include the Calvin cycle pathway, ATPase synthesis pathway, and genes related to electron transport, light response, and C₄ photosynthetic pathways. In total, the inventors collected 124 photosynthesis-related genes. The promoter region was a segment from 1000 bp upstream to 500 bp downstream of the transcription initiation site. The sequence of the promoter region was downloaded from the Arabidopsis database (Phytozome database).

Then, the transcription factors of all plants and the corresponding Position Weight Matrices (PWMs) were collected from the TRANSFAC database. A total of 124 transcription factors and corresponding PWMs were obtained. The ability of transcription factors to interact with candidate genes was predicted by constructing a transcription factor binding ability prediction algorithm (TRAP). The results showed that the mEmBP1 gene could interact with 43 downstream photosynthetic genes. Fisher's test reached a very significant level (P<0.001). The mEmBP1 gene is a maize-derived gene. After full-length amplification of the gene sequence in the present disclosure, it is transformed into rice and Arabidopsis, and its effect on photosynthetic genes and morphological characteristics are investigated.

### EXAMPLE 3. PROTEIN EXPRESSION POSITION OF MEMBP1 GENE AND GENERATION OF TRANSGENIC LINES

1. mEmBP1 Gene Localization

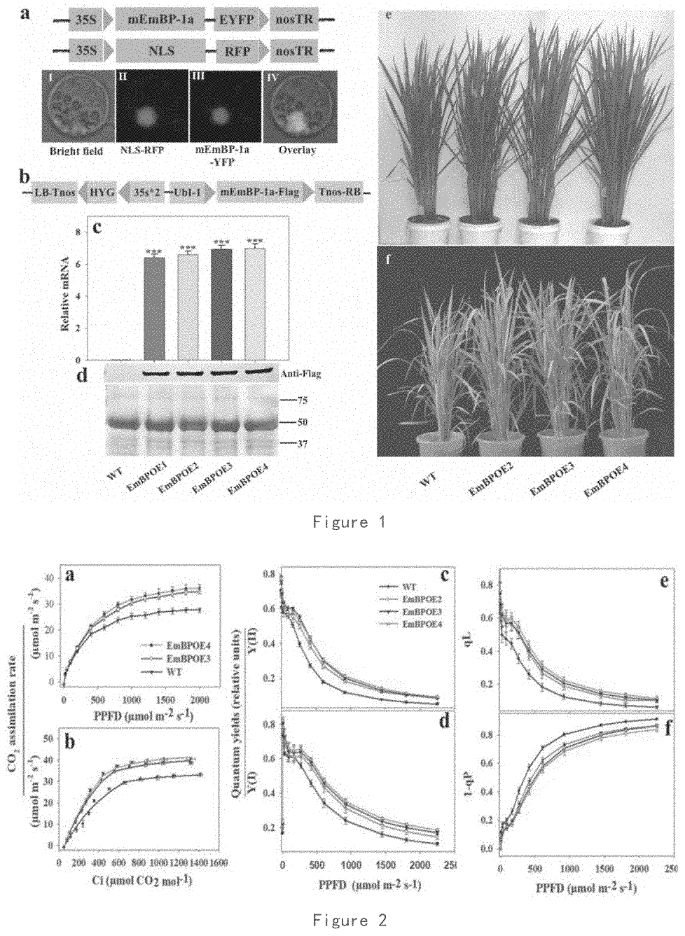

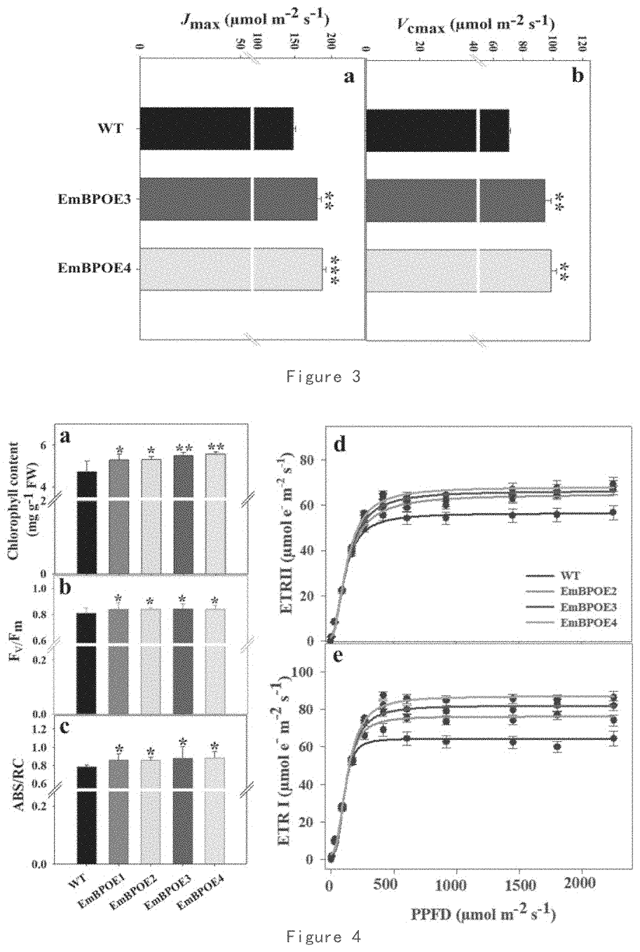

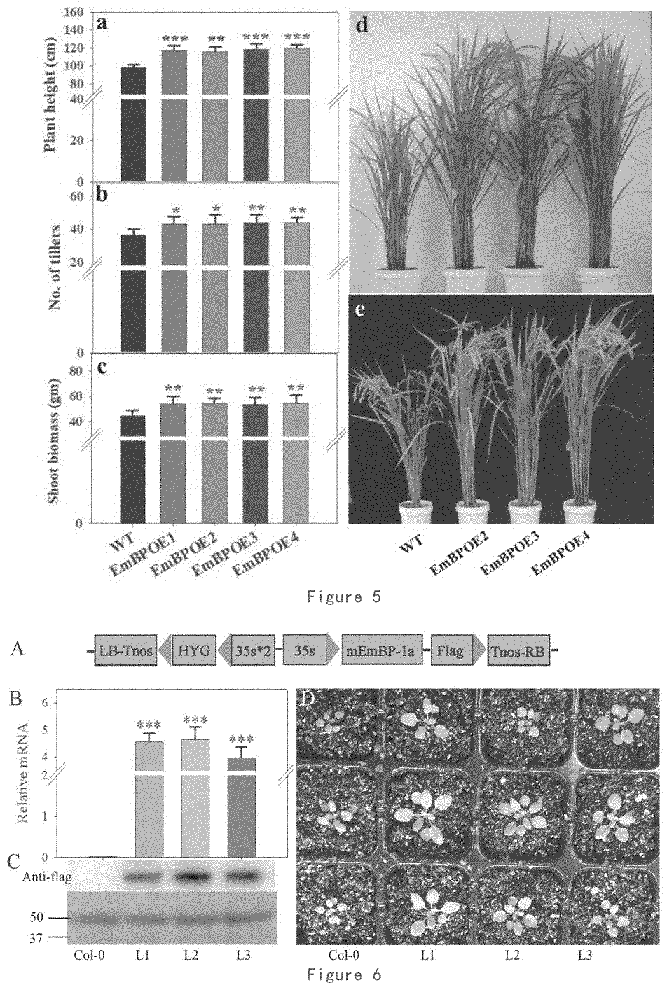

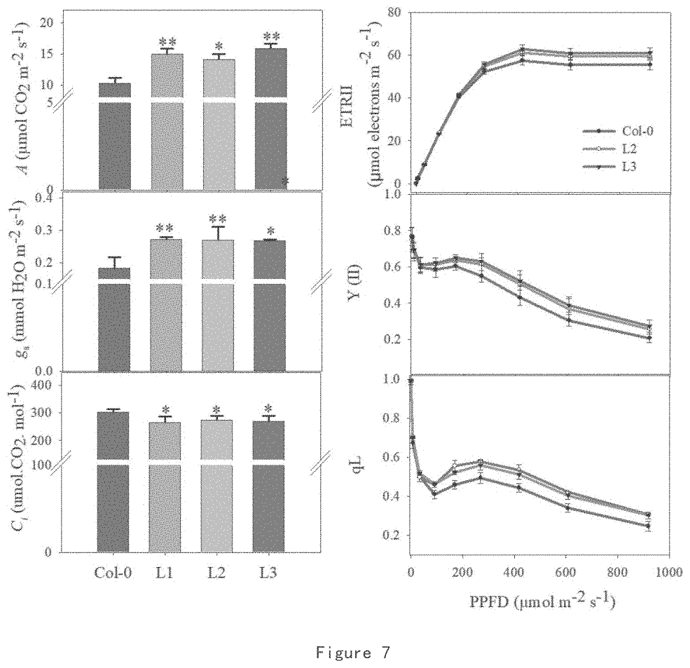

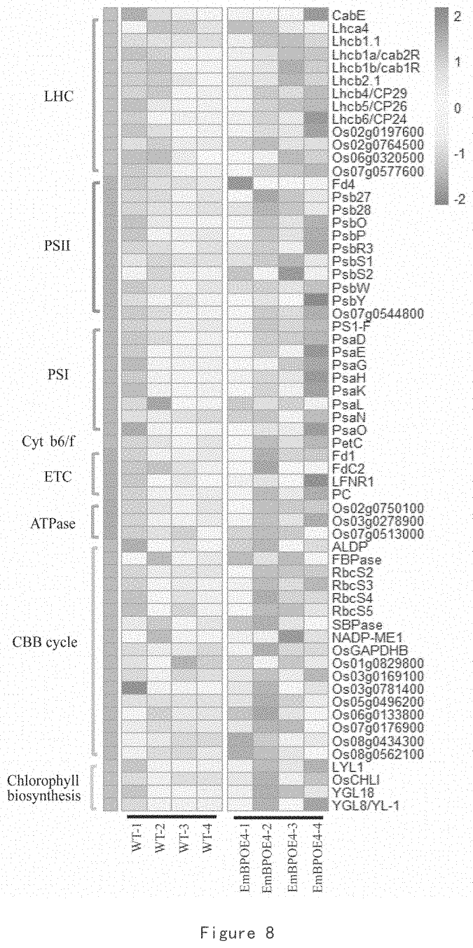

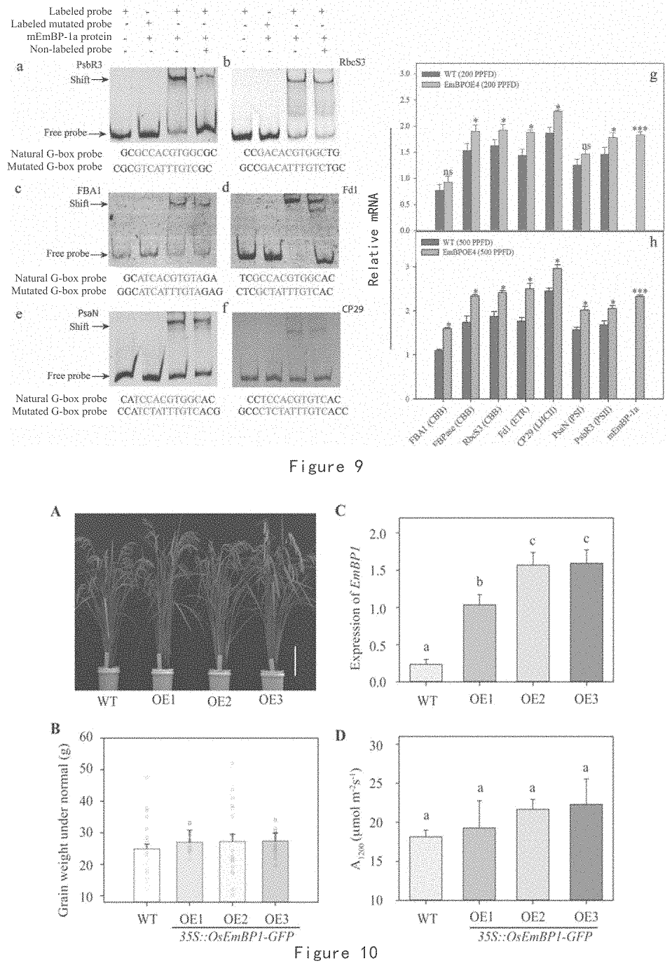

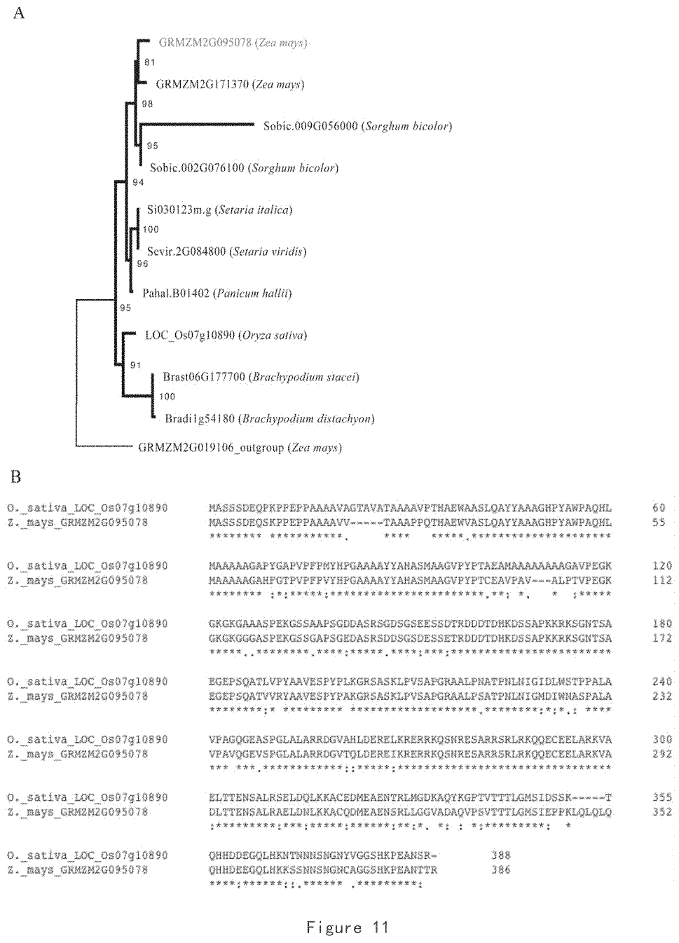

In order to verify the expression position of the encoded protein of the mEmBP1 gene, subcellular localization was analyzed and two vectors were constructed respectively (the upper panel of FIG. 1a), EmBP-1a (mEmBP1) linked to the YFP tag and the known nuclear encoding gene NLS linked to the RFP tag. Both genes were driven by the 35S strong promoter.

As shown in the lower panel of FIG. 1a, the spatial expression of EmBP-1a gene and NLS are completely coincident, indicating that mEmBP1 gene is localized in the nucleus.

2. Analysis of Plants Overexpressing mEmBP1 Gene

A second vector was constructed, EmBP-1a linked to Flag tag (FIG. 1b), and transformed into Nipponbare.

The results showed that there was a very high level of up-regulated expression among the three strains of the T3 generation, and the Flag immunoassay also proved that the mEmBP1 protein was highly up-regulated (FIGS. 1c-d).

FIGS. 1e-f shows the field performance of the three lines at different places (Shanghai Songjiang Breeding Base and Hainan Lingshui Breeding Base) at the peak tillering stage. Visually visible increases in plant height, in tiller number, and in biomass were occurred.

Table 4 shows the field yield survey of the rice materials overexpressing the maize mEmBP1 gene and the wild type planted in Shanghai Songjiang Breeding Base.

TABLE 4

| | spike number | | spikelet | grain number | thousand kernel | Yield | Increased |
|---|---|---|---|---|---|---|---|
| Genotype | per plant | spike length | per plant | per plant | weight (gm) | (gm/plant) | yield (%) |
| WT | 27.6 ± 1.85 | 24.84 ± 0.56 | 98.2 + 6.02 | 1314.6 ± 170.6 | 23.5 ± 1.6 | 31.4 ± 2.33 | |
| EmBPOE1 | 35.8 ± 1.96* | 24.34 ± 0.5^{ns} | 121.4 ± 5.13* | 1475.2 ± 121.8* | 26.2 + 1.0ns | 38.5 ± 1.30* | 22.6% |
| EmBPOE2 | 34.9 ± 2.15* | 24.4 ± 0.41^{ns} | 124.8 ± 5.68* | 1500.8 ± 234.6* | 27.1 ± 0.5^{ns} | 39.8 ± 2.83* | 26.8% |
| EmBPOE3 | 35.4 ± 1.75* | 24.66 ± 0.5 1^{ns} | 125.4 ± 7.00** | 1520.4 ± 216.3** | 27.5 + 1.1* | 40.6 + 2.60** | 29.3% |
| EmBPOE4 | 36.2 ± 1.64* | 24.3 ± 0.18^{ns} | 126.9 ± 4.33** | 1515.3 + 119.3** | 27.0 + 0.4^{ns} | 40.4 + 2.46** | 28.6% |

The values in the table represent mean±SE (n=15);

Statistical analysis were performed according to Students' T-test, *P≤0.05; **P≤0.01; ***P≤0.001.

According to the above results, spike number per plant, spikelet per plant, grain number per plant, thousand kernel weight and yield of the mEmBP1 gene-overexpressing plants significantly increases as compared with that of the wild type. The rice has achieved a significantly increased production in field environment.

### EXAMPLE 4. CHANGES IN PHOTOSYNTHETIC PHYSIOLOGICAL PARAMETERS OF TRANSGENIC LINES

In this example, the photosynthetic physiological parameters of three mEmBP1 transgenic lines and wild-type Nipponbare were compared.

It was found that the mEmBP1 transgenic line had higher photosynthetic efficiency under the conditions of specific light intensity and intracellular CO₂ concentration (FIGS. 2a-b). The quantum efficiencies of photosystem I and photosystem II were better in transgenic lines (FIGS. 2c-d). In addition, transgenic lines had higher levels of photoquenching and Qa reduction states (FIGS. 2e-f).

The inventors also found that in the mEmBP1 transgenic line, both Vcmax (Rubisco maximum catalytic efficiency) and the maximum electron transfer rate were significantly higher than those of the wild type (FIG. 3).

In addition, the chlorophyll fluorescence parameters in the mEmBP1 transgenic lines were also investigated to better reflect the leaf photosynthetic physiological indicators. It is found that the content of chlorophyll a+b, the maximum quantum yield (Fv/Fm), the aperture beam size of the reaction center (ABS/RC) and the electron transport chain (photosynthetic system I and photosynthetic system II) showed higher levels as compared with the wild type (FIGS. 4a-e).

### EXAMPLE 5. PERFORMANCE OF MEMBP1 TRANSGENIC LINES AT DIFFERENT GROWTH STAGES UNDER FIELD CONDITIONS

In order to study the morphological differences between mEmBP1 transgenic rice and wild-type Nipponbare rice materials, the performance of mEmBP1 lines at 70 days (late flowering) and 95 days (late grain filling) after emergence was analyzed.

The results showed that the transgenic lines had significantly increased plant height, tiller number and above-ground biomass (FIGS. 5a-c).

FIGS. 5d-e shows the field performance of two growth periods (70 days and 90 days after emergence, respectively). It can be seen that the plant height of mEmBP1 gene overexpressing plants is significantly higher than that of wild-type plants, and the tiller number and above-ground biomass are also significantly increased.

### EXAMPLE 6. ANALYSIS OF DIFFERENCE IN THE PHENOTYPE AND PHOTOSYNTHETIC PHYSIOLOGY PARAMETERS OF ARABIDOPSIS THALIANA OVEREXPRESSING MAIZE EMBP1 GENE

The inventors explored the physiological function of mEmBP1 gene in different species, and constructed a maize-derived mEmBP1 gene that is induced and expressed by 35s promoter (FIG. 6A).

The results showed that, compared with wild-type col, both the mEmBP1 gene and the encoded protein showed higher expression levels in transgenic lines (FIGS. 6B-C). The overexpressing transgenic lines simultaneously grown under climatic chamber conditions exhibited higher biomass accumulation (FIG. 6D).

### EXAMPLE 7. ANALYSIS OF DIFFERENCE IN THE PHENOTYPE AND PHOTOSYNTHETIC PHYSIOLOGY PARAMETERS OF ARABIDOPSIS THALIANA OVEREXPRESSING MAIZE EMBP1 GENE

The photosynthetic physiological parameters of Arabidopsis overexpressing the maize EmBP1 gene were also investigated, including photosynthetic efficiency (A), stomatal conductance (gs), intercellular CO₂ concentration (Ci), and chlorophyll fluorescence parameters including photosynthetic system II electrons transport rate (ETR), photosystem II efficiency (YII), and QA redox state (qL).

The results showed that the transgenic Arabidopsis lines exhibited better photosynthetic parameters described above (FIG. 7).

### EXAMPLE 8. TRANSCRIPTOMIC ANALYSIS OF MEMBP1 TRANSGENIC LINES AND WILD-TYPE NIPPONBARE

To identify which photosynthetic genes expression is affected by the mEmBP1 gene, genome-wide mRNA expression levels between different rice lines were analyzed (FIG. 8).

The results showed that a total of 65 photosynthesis-related genes were differentially expressed in mEmBP1 transgenic lines and wild-type Nipponbare, and were respectively enriched in LHC, PSII, PSI, Cyt b6f, ETC, ATPase, CBB cycle, Chlorophyll biological pathways.

### EXAMPLE 9. ANALYSIS OF THE INTERACTION BETWEEN MEMBP1 AND KEY PHOTOSYNTHETIC GENES

Among the 65 photosynthetic genes described above, 20 genes have G-BOX regulatory sequences in their promoter regions. qPCR results showed that 7 genes were significantly different in overexpressing and wild-type Nipponbare. In order to further confirm the binding relationship between mEmBP1 and the 7 photosynthetic genes, electrophoretic mobility shift assay (EMSA) was conducted.

Results of Electron mobility shift assay (FIGS. 9a-f) showed that mEmBP1 has strong interaction ability with 7 photosynthetic genes, including PsbR3, RbcS3, FBA1, FBPse, Fd1, PsaN and CP29. mEmBP-1a binds to the G-Box motif of the target genes in photosynthesis. In order to analyze the expression changes of the 7 genes under different light conditions, light intensities of 200PPFD and 500PPFD were selected as low-light and weak-light conditions, respectively.

The results showed that these genes showed difference under low light and weal light, that is, provided that the EmBP-1a gene was significantly up-regulated in the EmBP-1a line, other genes also showed 10-20% different degrees of increase.

This result shows that EmBP1 has the ability to interact with key photosynthetic genes, can affect photosynthetic efficiency genes on the whole, adapt to different light environments, and can better perform photosynthesis in different light environments, which is beneficial to plant growth and development. Meanwhile, EmBP1 can effectively improve the electron transfer efficiency.

### EXAMPLE 10. OVEREXPRESSION OF RICE-DERIVED EMBP1 IN RICE

The amino acid sequence of rice EmBP1 (OsEmBP1) protein is as follows (SEQ ID NO:3):

>LOC_Os07gl0890.1
MASSSDEQPKPPEPPAAAAVAGTAVATAAAAVPTHAEWAASLQAYYAAA
GHPYAWPAQHLMAAAAAGAPYGAPVPFPMYHPGAAAAYYAHASMAAGVP
YPTAEAMAAAAAAAAGAVPEGKGKGKGAAASPEKGSSAAPSGDDASRSG
DSGSEESSDTRDDDTDHKDSSAPKKRKSGNTSAEGEPSQATLVPYAAVE
SPYPLKGRSASKLPVSAPGRAALPNATPNLNIGIDLWSTPPALAVPAGQ
GEASPGLALARRDGVAHLDERELKRERRKQSNRESARRSRLRKQQECEE
LARKVAELTTENSALRSELDQLKKACEDMEAENTRLMGDKAQYKGPTVT
TTLGMSIDSSKTQHHDDEGQLHKNTNNNSNGNYVGGSHKPEANSR*

Rice EmBP1 CDS sequence is as follows (SEQ ID NO:4):

>LOC_Os07gl0890.1
ATGGCGTCCTCGTCGGACGAGCAGCCGAAGCCGCCGGAGCCGCCCGCGG
CGGCGGCGGTGGCGGGGACGGCCGTGGCCACCGCCGCCGCGGCGGTGCC
GACGCACGCCGAGTGGGCGGCTTCGCTGCAGGCGTACTACGCCGCCGCG
GGGCACCCCTACGCGTGGCCCGCGCAGCATCTGATGGCGGCGGCGGCTG
CGGGGGCGCCGTACGGCGCGCCGGTGCCGTTCCCGATGTACCACCCGGG
CGCCGCCGCGGCGTACTACGCGCACGCGTCCATGGCCGCGGGTGTTCCT
TACCCGACAGCTGAAGCCATGGCGGCGGCGGCGGCGGCGGCGGCGGGGG
CGGTGCCGGAAGGGAAGGGGAAGGGGAAGGGCGCCGCCGCGTCGCCTGA
GAAGGGAAGCTCCGCGGCGCCCTCTGGGGATGATGCATCCCGGAGTGGT
GACAGTGGCAGCGAGGAGTCGTCTGATACTAGAGATGATGACACTGACC
ACAAGGATTCGTCTGCACCTAAGAAAAGGAAATCTGGTAATACATCGGC
AGAAGGTGAGCCGTCTCAAGCTACGCTTGTGCCCTATGCTGCTGTCGAG
TCACCGTATCCGTTGAAGGGGAGGTCTGCGTCGAAGCTTCCAGTTTCTG
CACCAGGGCGGGCGGCACTTCCTAATGCCACACCTAATTTGAACATAGG
GATAGATCTTTGGAGTACTCCCCCAGCCTTAGCTGTGCCCGCAGGGCAG
GGGGAAGCAAGTCCTGGGTTGGCACTTGCTCGACGTGATGGTGTTGCTC
ACCTGGATGAGCGTGAATTGAAGAGGGAGAGGCGCAAACAATCTAACAG
AGAGTCTGCCAGGAGATCAAGGTTGCGCAAGCAGCAAGAGTGTGAGGAA
CTAGCTCGGAAGGTTGCTGAACTGACAACTGAGAACAGTGCCCTTCGGT
CAGAGCTTGATCAGCTTAAGAAGGCCTGTGAGGATATGGAAGCAGAGAA
TACACGACTGATGGGTGATAAGGCTCAATACAAGGGACCAACTGTGACA
ACCACTCTGGGTATGAGCATCGACTCATCGAAGACGCAACACCATGACG
ACGAGGGCCAGCTTCACAAGAACACTAATAATAACAGCAACGGGAACTA
TGTAGGTGGCAGCCACAAACCAGAGGCTAACTCTAGGTGA

The encoding gene of rice EmBP1 was inserted into the BamHI/SacI site of pCAMBIA1301 (containing the GFP tag, expressed under the control of the CaMV 35S promoter, and containing the hygromycin B phosphotransferase (HPT) gene) (Youbio, China, VT1842) to obtain 35S::OsEmBP1-GFP (Os07g10890).

The transgenic rice overexpressing 35S::EmBP1 was prepared by Agrobacterium-mediated transformation using the expression vector.

The expression of rice EmBP1 in rice was identified with the following primers:

(SEQ ID NO: 33)
Forward: GGAGTACTCCCCCAGCCTTA;
(SEQ ID NO: 34)
Reverse: TTGCGCAACCTTGATCTCCT.

As shown in FIG. 10C, the overexpressing rice plants showed higher expression of the EmBP1 gene than the wild type. The overexpressing plants were compared with wild type.

As shown in FIG. 10A, the overexpressed rice plants showed a phenotype of increased plant height, increased above-ground biomass, and increased tiller number.

As shown in FIG. 10B, the grain weight of the overexpressing rice plants was significantly increased.

As shown in FIG. 10D, the photosynthesis rate of overexpressing rice plants was significantly increased under A₁₂₀₀ light intensity.

### EXAMPLE 11. GENE CONSERVATION STUDY

The functions of the maize- and rice-derived EmBP1 genes are highly uniform, and the comparison shows that they have high sequence conservation. The sequence homology comparison is shown in FIG. 11B. This explains why they are functionally identical or similar in the previous results.

A phylogenetic tree for bZIP proteins EmBP-1 of different model plants based on the neighbor joining method was further established, as shown in FIG. 11A. It can be seen that there is a high degree of conservation between maize (Zea mays) and sorghum (Sorghum bicolor), millet (Setaria italica), panicum (panicum hallii), rice (Oryza sativa), brachypodium stacei, and Brachypodium distachyon. Therefor, their functions are the same or similar.

Each reference provided herein is incorporated by reference to the same extent as if each reference was individually incorporated by reference. In addition, it should be understood that based on the above teaching content of the disclosure, those skilled in the art can practice various changes or modifications to the disclosure, and these equivalent forms also fall within the scope of the appended claims.

1. (canceled)

2. (canceled)

3. A method of improving agronomic traits of plants or preparing plants with improved agronomic traits, comprising: increasing the expression or activity of EmBP1 in plants;
wherein the improved agronomic traits include: (i) increasing photosynthesis efficiency, (ii) regulating the expression of photosynthetic genes, (iii) increasing yield, (iv) increasing biomass, (v) increasing plant height, (vi) increasing tiller number;
wherein, the EmBP1 includes its homologues.

4. The method according to claim 3, wherein, increasing the expression or activity of EmBP1 includes:
regulating with an up-regulated molecule that interacts with EmBP1, thereby increasing the expression or activity of EmBP1;
or overexpressing EmBP1 in plants.

5. The method according to claim 3, wherein, the plant includes a plant selected from the following group, or the EmBP1 is from a plant selected from the following group:
Gramineae, Brassicaceae, Solanaceae, Leguminosae, Cucurbitaceae, asteraceae, Salicaceae, Moraceae, Myrtaceae, Lycopodiaceae, Selaginellaceae, Ginkgoaceae, Pinaceae, Cycadaceae, Araceae, Ranunculaceae, Platanaceae, Ulmaceae, Juglandaceae, Betulaceae, Actinidiaceae, Malvaceae, Sterculiaceae, Tiliaceae, Tamaricaceae, Rosaceae, Crassulaceae, Caesalpinaceae, Fabaceae, Punicaceae, Nyssaceae, Cornaceae, Alangiaceae, Celastraceae, Aquifoliaceae, Buxaceae, Euphorbiaceae, Pandaceae, Rhamnaceae, Vitaceae, Anacardiaceae, Burseraceae, Campanulaceae, Rhizophoraceae, Santalaceae, Oleaceae, Scrophulariaceae, Pandanaceae, Sparganiaceae, Aponogetonaceae, Potamogetonaceae, Najadaceae, Scheuchzeriaceae, Alismataceae, Butomaceae, Hydrocharitaceae, Triuridaceae, Cyperaceae, Palmae(Arecaceae), Araceae, Lemnaceae, Flagellariaceae, Restionaceae, Centrolepidaceae, Xyridaceae, Eriocaulaceae, Bromeliaceae, Commelinaceae, Pontederiaceae, Philydraceae, Juncaceae, Stemonaceae, Liliaceae, Amaryllidaceae, Taccaceae, Dioscoreaceae, Iridaceae, Musaceae, Zingiberaceae, annaceae, Marantaceae, Burmanniaceae, Chenopodiaceae, or Orchidaceae.

6. The method according to claim 5, wherein, said Gramineae are selected from: rice, maize, sorghum, millet, panicum, wheat, barley, oat, rye, brachypodium stacei, and brachypodium;
said Brassicaceae are selected from: rape, Chinese cabbage, Arabidopsis;
said Malvaceae are selected from: cotton, Hibiscus rosa-sinensis, Hibiscus;
said Leguminosae are selected from: soybean, Alfalfa;
said Solanaceae include: tobacco, tomato, pepper;
said Cucurbitaceae include: pumpkin, watermelon, cucumber;
said Rosaceae include: apple, peach, plum, begonia;
said Chenopodiaceae are selected from: sugar beet;
said Asteraceae include: sunflower, lettuce, asparagus, Artemisia apiacea, Jerusalem artichoke, Stevia rebaudiana;
said Salicaceae include: poplar, willow;
said Myrtaceae include: eucalyptus, clove, myrtle;
said Euphorbiaceae include: rubber tree, cassava, castor;
said Fabaceae include: peanut, pea, Astragalus membranaceus.

7. The method according to claim 5, wherein, the plant isi Gramineae, and increasing yield or biomass includes: increasing seed weight, increasing seed number, increasing the weight of seeds, increasing spike number, increasing spikelet number, or increasing spike length.

8. The method according to claim 3, wherein, regulating the expression of photosynthetic genes includes up-regulating the expression of photosynthetic genes.

9. The method according to claim 8, wherein, the photosynthetic genes include photosynthetic genes involved in LHC, PSII, PSI, Cyt b6f, ETC, ATPase, CBB cycle and/or Chlorophyll biological pathway.

10. The method according to claim 3, wherein, improving photosynthetic efficiency includes: increasing CO₂ absorption rate, increasing electron transfer efficiency, increasing maximum electron transfer rate, increasing Rubisco maximum catalytic efficiency, increasing chlorophyll content, increasing maximum quantum yield, increasing the aperture beam size of the reaction center, or improving the level of the electron transport chain.

11. The method according to claim 3, wherein, the amino acid sequence of the EmBP1 is selected from the group consisting of:
(i) a polypeptide having the amino acid sequence shown in SEQ ID NO: 1;
(ii) a polypeptide derived from (i) and having one or more amino acids deleted, substituted, or inserted in the amino acid sequence of SEQ ID NO: 1, and still having said function of regulating agronomic traits;
(iii) a polypeptide with an amino acid sequence having ≥80% homology to SEQ ID NO:1, and still having said function of regulating agronomic traits; or
(iv) an active fragment of the polypeptide of the amino acid sequence shown in SEQ ID NO: 1.

12. The method according to claim 3, wherein, the nucleotide sequence of the EmBP1 gene is selected from the group consisting of:
(a) a polynucleotide encoding the polypeptide shown in SEQ ID NO: 1; ((b) a polynucleotide of the sequence shown in SEQ ID NO: 2;
(c) a polynucleotide with a nucleotide sequence having ≥80% homology to SEQ ID NO:2;
(d) a polynucleotide formed by truncating or adding 1-60 nucleotides at the 5′ end and/or 3′ end of the polynucleotide shown in SEQ ID NO: 2;
(e) a polynucleotide complementary to any of (a)-(d).

13. A plant cell expressing exogenous EmBP1 or its homologue, or an expression cassette comprising exogenous EmBP1 or its homologue, the expression cassette comprises: promoter, gene encoding EmBP1 or its homologue, terminator.

14. (canceled)

15. A method for targeted selection of plants with improved agronomic traits, the method comprising:
identifying the expression or activity of EmBP1 in a test plant, if the expression or activity of EmBP1 in the test plant is higher than the average value of the expression or activity of EmBP1 in such plants, then it is an plant with improved agronomic traits;
wherein, the improved agronomic traits include: (i) photosynthetic efficiency, (ii) expression of photosynthetic genes, (iii) yield, (iv) biomass, (v) plant height, (vi) tiller number;
wherein, the EmBP1 includes its homologues.

16. The method according to claim 8, wherein the EmBP1 or homologue thereof regulates (including up-regulates) the expression of photosynthetic genes by regulating the promoter of the photosynthetic gene.

17. The method according to claim 8, wherein EmBP1 or homologue thereof binds to the G-box of the promoter.

18. The method according to claim 9, wherein the photosynthetic genes include PsbR3, RbcS3, FBA1, FBPse, Fd1, PsaN and/or CP29.

19. The plant cell according to claim 13, wherein the expression cassette is contained in a construct or an expression vector.
